(19) United States
(12) Patent Application Publication
(10) Pub. No.: US 2010/0177904 A1
(43) Pub. Date: Jul. 15, 2010

(54) Noise Reducing Earphone

(76) Inventors: Po-Hsun Sung, Taichung (TW); Cheng-Ho Tsai, Taichung (TW); Chiung-Wen Yeh, Taichung (TW); Hong-Ching Her, Taichung (TW)

Correspondence Address: HDLS Patent & Trademark Services, P.O. BOX 220746, CHANTILLY, VA, 20153-0746, US

(21) Appl. No.: 12/352,897

(22) Filed: Jan. 13, 2009

(51) Int. Cl. A61F 11/06

(52) U.S. Cl. 381/71.6

(57) ABSTRACT

A noise reducing earphone includes a body, a speaker, a microphone and a signal processor. The body includes a cavity and a sound tunnel. The speaker and the microphone are both disposed in the cavity. The speaker is configured for outputting audio signals through the sound tunnel. The microphone is disposed besides the speaker and configured for receiving the audio signals in the cavity. The signal processer is electrically connected to the speaker and the microphone and configured for providing an electrical signal to the speaker so that the electrical signal is converted to an audio signal by the speaker. The microphone is configured for converting the received audio signals to an electrical signal, and sending the electrical signal to the signal processor. The signal processor is configured for outputting to the speaker an anti-phase signal with respect to the noise component defined by the signal processor in the audio signal.

### BACKGROUND

1. Field of the Invention

The present invention relates to a noise reducing earphone, and more particularly to an in-ear noise reducing earphone that provides a relatively good output frequency response.

2. Description of Related Art

A current noise reducing earphone usually has at least a speaker and a microphone disposed inside the earphone. The microphone receives sound waves of noise coming from the outside, converts the sound waves into a noise signal and sends the signal to a noise reducing circuit. The noise reducing circuit generates an anti-phase signal with respect to the noise signal and sends the anti-phase signal to the speaker. According to the anti-phase signal, the speaker generates sound waves that are anti-phase with the noise sound waves and interfere with the noise sound waves so as to achieve noise reduction.

Referring to FIG. 1A of a schematic of a conventional in-ear noise reducing earphone, the in-ear noise reducing earphone mainly includes a earphone housing 10, an earplug 11, a speaker 12, a microphone 13 and a noise reducing circuit 14. The earplug 11 is covered on an opening end of the earphone housing 10. The combination of the earplug 11 and the earphone housing 10 can accommodate the speaker 12, the microphone 13 and the noise reducing circuit 14. The speaker 12 and the microphone 13 are respectively connected to the noise reducing circuit 14 by a wire 15. From the perspective view along the direction of the opening end of the earplug 11 being plugged into a user's ear canal, the microphone is located in front of the speaker 12. In other words, the microphone 13 is located between the speaker 12 and the ear canal. With such configuration, the sound waves transmitted to the ear canal from the speaker 12 can be received by the microphone first and directly processed by the noise reducing circuit 14 before being output by the speaker 12. This is called feedback active noise cancelling technology. It can be used to monitor the noise signal eventually entering the user's ears and thus can achieve better performance relative to noise reduction.

The above-mentioned feedback active noise cancelling technology is actually used frequently in earphones with ear muff type or on ear type, in which cases, because the cavity formed between the earphone and the ear canal is relatively large, the output frequency response curve of the earphones can still roughly match the frequency response curve of the earphone without the noise reduction technology being introduced. However, when applying the feedback active noise cancelling technology to in-ear earphones, because the cavity formed between the earphone and the ear canal is smaller, the output frequency response curves become significantly different. Referring to FIG. 1B, showing a frequency response curve S1 of the noise reducing earphone depicted in FIG. 1A, relatively significant resonance decay (a significant drop) can be found in the frequency range of 3000 Hz to 6000 Hz on the frequency response curve S1 of the conventional in-ear noise reducing earphone. Although the audible range of human is normally 20 Hz to 20000 Hz, the sensitive audible frequency range is between 2000 Hz to 3000 Hz. The output frequency of musical instruments can be as high as 4000 Hz. This means that the high frequency range of 3000 Hz to 6000 Hz where the frequency response curve S1 of the conventional in-ear noise reducing earphone decays is close to or even overlapping with the sensitive audible frequency range. Hence it is evident that the sound output performance of the conventional in-ear noise reducing earphone is sacrificed by the positioning of the microphone and such sacrifice is generally not desired by the user.

### BRIEF SUMMARY

In view of the foregoing, an objective of the present invention is to provide a noise reducing earphone that reduces noise and has a relative good output frequency response.

To achieve the objective, the present invention provides a noise reducing earphone that includes a body, a speaker, a microphone and a signal processor. The body includes a cavity and a sound tunnel. The speaker and the microphone are both disposed in the cavity. The speaker is configured for outputting audio signals through the sound tunnel. The microphone is disposed besides the speaker and configured for receiving the audio signals in the cavity. The signal processer is electrically connected to the speaker and the microphone and configured for providing an electrical signal to the speaker so that the electrical signal is converted to an audio signal by the speaker. The microphone is configured for converting the received audio signals to an electrical signal, and sending the electrical signal to the signal processor. The signal processor is configured for outputting to the speaker an anti-phase signal with respect to the noise component defined by the signal processor in the audio signal.

In addition, the present invention also has an opening formed on the edge of the cavity and by adjusting a damping element that is disposed on the opening the opening can be controlled to be open or closed. The damping element can be replaceable or movable, through which the extent of connection between the cavity and the outside can be adjusted.

As mentioned above, the present invention places the microphone besides the speaker, the distance between the speaker and the sound tunnel is greatly reduced and the high frequency decay that exists in the conventional noise reducing earphones is shifted to be in a higher frequency range that is beyond normal human audible range. By this means, the introduction of noise reduction functionality does not lead to sacrifice of high frequency performance of the earphone and instead gives the user a better hearing experience. In addition, an opening is formed on the edge of the cavity, and a damping element is used to control the opening to be open or closed so that the noise insertion loss presents a relatively large dB value in a relatively wide range of frequencies, which means noise reduction is achieved equally well at different frequencies in that wide frequency range. Hence, the overall noise reduction is enhanced.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1A is a schematic view of a conventional noise reducing earphone;

FIG. 1B shows a frequency response curve of the noise reducing earphone depicted in FIG. 1A;

FIG. 2 is a schematic cross-sectional view of a noise reducing earphone according to a first embodiment of the present invention;

FIG. 3 illustrates a comparison between the output frequency response curves of the noise reducing earphones depicted in FIG. 2 and in FIG. 1A;

FIG. 4 is a schematic cross-sectional view of a noise reducing earphone according to a second embodiment of the present invention;

FIG. 5A shows sensitivity frequency response curves of the noise reducing earphone depicted in FIG. 4;

FIG. 5B shows phase frequency response curves of the noise reducing earphone depicted in FIG. 4;

FIG. 5C shows noise insertion loss frequency response curves of the noise reducing earphone depicted in FIG. 4; and

FIG. 6 is a schematic cross-sectional view of a noise reducing earphone according to a third embodiment of the present invention.

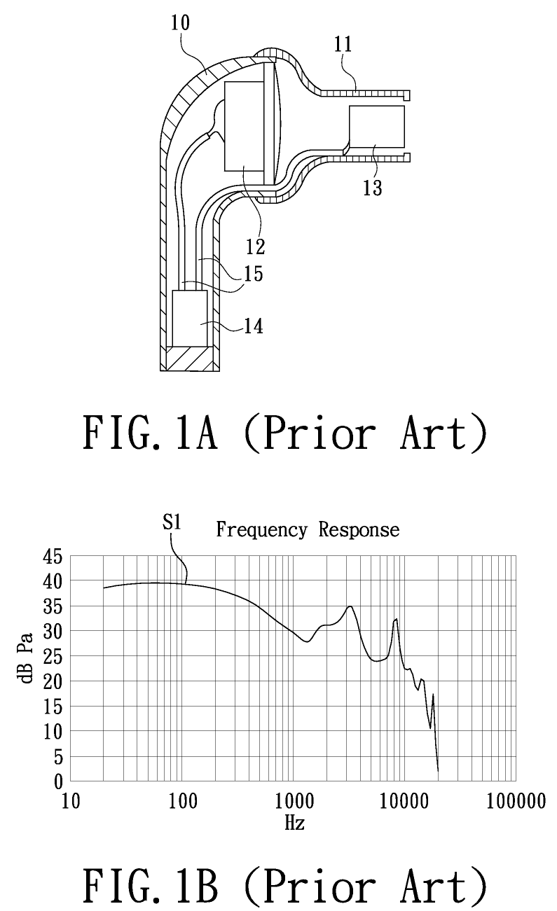

### DETAILED DESCRIPTION

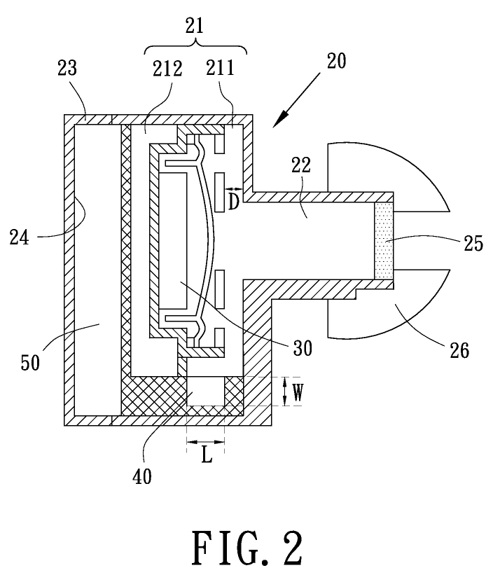

Referring to FIG. 2, a noise reducing earphone according to a first embodiment of the invention is provided. The noise reducing earphone includes a body 20, a speaker 30, a microphone 40 and a signal processor 50. The body 20 includes a cavity 21 and a sound tunnel 22. The cavity 22 is connected to the outside through the sound tunnel 22. The speaker 30 and the microphone 40 are both disposed in the cavity 21. The speaker 30 is positioned in the way that it divides the cavity 21 into a front cavity 211 and a rear cavity 212. The microphone 40 is disposed in the front cavity 211 and besides the speaker 30. The signal processor 50 is electrically connected to the speaker 30 and the microphone 40, and configured for providing an electrical signal to the speaker 30 so that the electrical signal is converted to an audio signal and the audio signal is output from the sound tunnel 22. The microphone 40 is configured for receiving the audio signal and other audio signals for example echoes generated from the canal in the cavity 21, converting the audio signals to an electrical signal, and sending the electrical signal to the signal processor 50. The signal processor 50 is configured for outputting to the speaker 30 a signal that is anti-phase with the noise component defined by the signal processor in the audio signal.

It is noted that in this embodiment because the microphone 40 is disposed besides the speaker 30, the distance D between the speaker 30 and the sound tunnel 22 can be designed to be less than the diameter L of the microphone 40, or even less than the thickness W of the microphone 40.

In addition, in this embodiment, the body 20 further includes a back cover 23. The back cover 23 is attached to the cavity 21, thereby forming a containing space 24 for accommodating the signal processor 50. Further, a mask 25 is disposed at the opening end of the sound tunnel 22 for preventing dust and dirt from entering the sound tunnel 22. Furthermore, an earplug 26 is covered on the opening end of the sound tunnel 22. The earplug 26 is made of soft materials such as rubber so as to be conveniently and comfortably plugged into a user's ear canal.

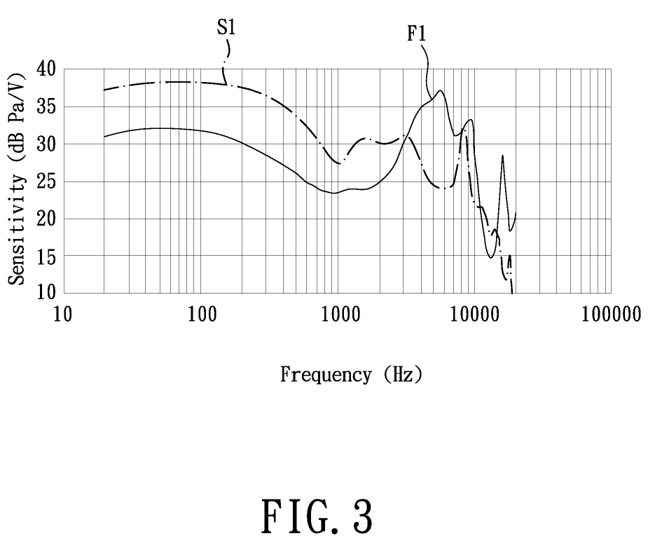

Referring to FIG. 3, which illustrates a comparison between the output frequency response curves of the noise reducing earphone according to the first embodiment of the present invention and the conventional noise reducing earphone, wherein the horizontal axis represents frequency and the vertical axis represents sensitivity. In the first embodiment, the microphone 40 is disposed besides the speaker 30. As a result, the speaker 30 can be disposed at a position very close to the position of the sound tunnel 22. In this way, the microphone 40 in the cavity 21 can directly receive sound output by the speaker 30 without standing in the path between the speaker 30 and the sound tunnel 22 and causing interference. Hence in FIG. 3, it shows that compared to the frequency response curve S1 of the conventional noise reducing earphone, the decay region of the frequency response curve F1 of the noise reducing earphone according to the first embodiment of the present invention shifts to be after 10000 Hz. Because 10000 Hz and higher frequencies are already substantially beyond the audible range of ordinary people, the audible effect of the decay is minimized.

It must be noted that although the relatively good frequency response shown in FIG. 3 is measured in the case wherein the distance D between the speaker 30 and the sound tunnel 22 is less than the thickness W of the microphone 40, as long as the microphone is designed not to block in the sound outputting path of the speaker 30, there is a similar effect to some extent. It is understood that by designing the distance D between the speaker 30 and the sound tunnel 22 to be less than the diameter L of the microphone 40, the effect of shifting the decay region to higher frequencies can also be achieved.

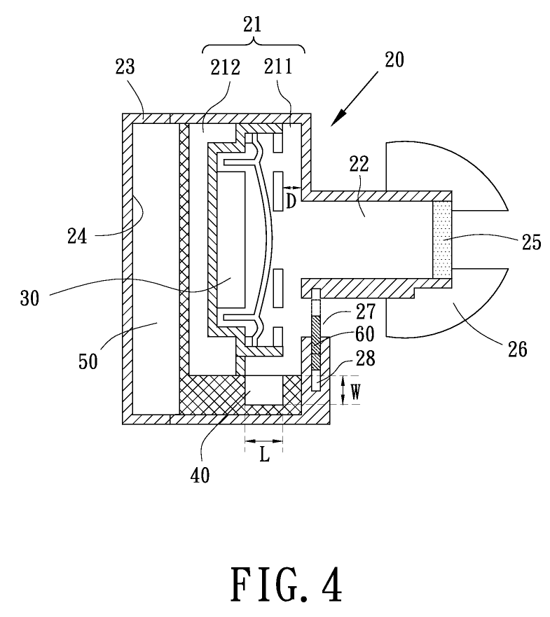

Referring to FIG. 4, a noise reducing earphone according to a second embodiment of the invention is provided. The noise reducing earphone includes a body 20, a speaker 30, a microphone 40 and a signal processor 50. The difference between this embodiment and the first embodiment of the invention is that in this embodiment an opening 27 is formed on the edge of the cavity 21 of the body 20. As shown in FIG. 4, the opening 27 can be formed close to the sound tunnel 22. A channel 28 is formed on the cavity 21 corresponding to the opening 27 for accommodating a damping element 60. By adjusting the damping element 60, the opening 27 can be controlled to be open or closed, and the extent of connection between the cavity 21 and the outside can be adjusted. For example, the opening 27 can be adjusted to be fully open, fully closed or partially open.

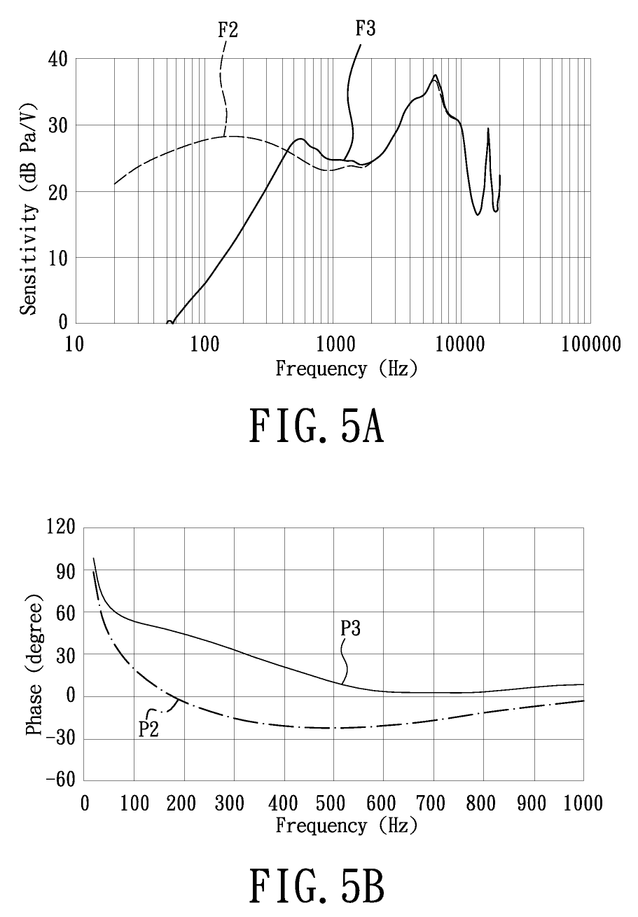

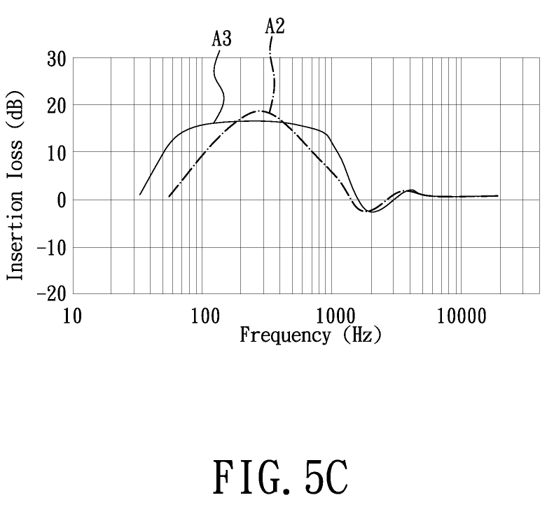

FIGS. 5A to 5C illustrate the frequency responses of sensitivity, phase and noise insertion loss of the noise reducing earphone according to the second embodiment in different opening status. In FIG. 5A, when a user closes the opening 27 with the damping element 60 a sensitivity frequency response curve F2 is measured; when the user moves the damping element 60 and opens the opening 27, another sensitivity frequency response curve F3 is measured. In FIG. 5B, when the opening 27 is closed, a signal that the microphone 40 receives has a phase frequency response curve P2; when the opening 27 is open, a signal that the microphone 40 receives has a phase frequency response curve P3. In FIG. 5C, when the opening 27 is closed, a noise insertion loss frequency response curve A2 is measured in the cavity 21; when the opening 27 is open, a noise insertion loss frequency response curve A3 is measured in the cavity 21.

Referring to FIGS. 5A to 5C, when the opening 27 is controlled to switch from being closed to being open, the phase shift change of audio signals in the cavity 21 over the frequency turns more gradual. Correspondingly, the noise insertion loss frequency response curve turns from a sine wave shape to a close to trapezoid shape. In FIG. 5C, relatively large dB values cover a wide range of frequencies within a certain spectrum, which means noise reduction is achieved equally well in that wide frequency range. Hence, a fairly good overall noise reduction is achieved.

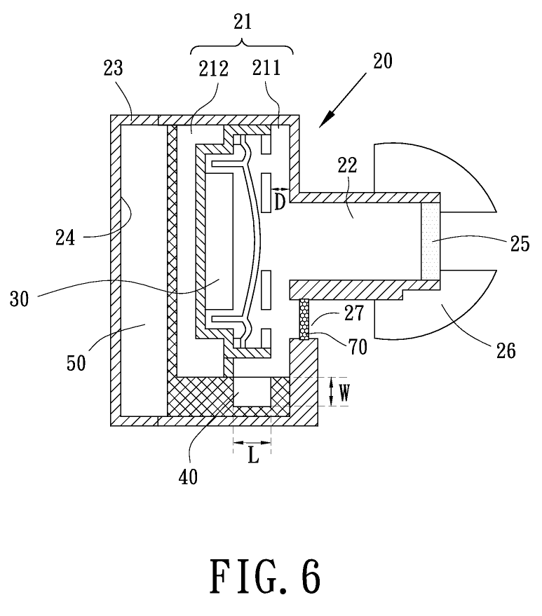

Referring to FIG. 6, a noise reducing earphone according to a third embodiment of the invention is provided. The noise reducing earphone includes a body 20, a speaker 30, a microphone 40 and a signal processor 50. The difference between this embodiment with the second embodiment of the invention is that a damping element 70 is directly disposed at the opening 27 of the body 20. The damping element 70 is replaceable and by replacing different damping elements 70 with different damping coefficients, different frequency responses can be achieved to meet different requirements. It is understood that the effect of replacing the damping element 70 is similar to moving the damping element 60 in the second embodiment of the present invention.

In the second and third embodiments of the invention, the extent of connection between the cavity 20 and the outside is controlled by the damping elements 60 and 70. From the curves in FIGS. 5A to 5C, it can be known a relatively good noise reduction is achieved when the damping element 60 turns the opening 27 from closed to open. In other words, if only the opening 27 but not the damping elements 60 and 70 are disposed on the cavity 20, the frequency response of the cavity 21 is still modified to deliver the same noise reduction as shown in the curve A3 in FIG. 5C.

It must be noted that although the speaker in the above embodiments are all dynamic speakers, replacing the dynamic speakers with balanced armature speakers in the embodiments will not change the effects the present invention can achieve.

In comparison with the prior art, the noise reducing earphone according to the embodiments of the present invention places the microphone besides the speaker, so that the distance between the speaker and the sound tunnel is greatly reduced and the high frequency decay that exists in the conventional noise reducing earphones is shifted to be in a higher frequency range that is beyond normal human audible range. By this means, the introduction of noise reduction functionality does not lead to sacrifice of high frequency performance of the earphone and gives the user a better hearing experience. In addition, an opening is formed on the edge of the cavity, and a damping element is used to control the opening to be open or closed. When the opening is controlled to switch from being closed to being open, the noise insertion loss presents a relatively large dB value in a wide range of frequencies within a certain spectrum, which means noise reduction is achieved equally well at different frequencies in that wide frequency range. Hence, a fairly good overall noise reduction is achieved.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1 A noise reducing earphone comprising:
a body having a sound tunnel and a cavity connected to outside through the sound tunnel;
a speaker disposed in the cavity for outputting audio signals;
a microphone disposed in the cavity and besides the speaker for receiving the audio signals in the cavity; and
a signal processor electrically connected to the speaker and the microphone and configured for generating an anti-phase noise cancelling signal according to the audio signals received by the microphone and transmitting the anti-phase noise cancelling signal to the speaker to be converted to a sound component and played out;
wherein the microphone is disposed beside the speaker so that the microphone receives audio signals without being in a position to directly interfere with the speaker outputting sound waves.

2. The noise reducing earphone of claim 1, wherein a distance between the speaker and the sound tunnel is less than a diameter of the microphone.

3. The noise reducing earphone of claim 1, wherein the distance between the speaker and the sound tunnel is less than a thickness of the microphone.

4. The noise reducing earphone of claim 1 further comprising a back cover attached to the cavity and thereby forming a containing space for accommodating the signal processor.

5. The noise reducing earphone of claim 1, wherein a mask is disposed at an opening end of the sound tunnel.

6. The noise reducing earphone of claim 1, wherein an earplug is covered on an opening end of the sound tunnel.

7. The noise reducing earphone of claim 1, wherein an opening is formed on an edge of the cavity for adjusting the frequency response of the cavity.

8. The noise reducing earphone of claim 7, wherein the opening is formed close to the sound tunnel.

9. The noise reducing earphone of claim 7 further comprising a damping element disposed at the opening.

10. The noise reducing earphone of claim 9, wherein a channel is formed on the cavity corresponding to the opening for accommodating the damping element, and the damping element is movable in the channel so as to control the opening to be open or closed.
